(19) United States
(12) Patent Application Publication
(10) Pub. No.: US 2025/0072776 A1
(43) Pub. Date: Mar. 6, 2025

(54) SENSOR UNIT AND TOILET SEAT DEVICE

(71) Applicant: TOTO LTD., Kitakyushu-shi (JP)

(72) Inventors: Masaya Nakata, Fukuoka (JP); Yoshiyuki Kaneko, Fukuoka (JP); Shotaro Wada, Fukuoka (JP); Motoki Kobayashi, Fukuoka (JP); Yusuke Motoda, Fukuoka (JP)

(21) Appl. No.: 18/786,673

(22) Filed: Jul. 29, 2024

(30) Foreign Application Priority Data
Aug. 31, 2023 (JP) 2023-140837
Aug. 31, 2023 (JP) 2023-141508

(51) Int. Cl. A61B 5/026; A61B 5/00

(52) U.S. Cl. CPC A61B 5/0261; A61B 5/6887

(57) ABSTRACT

A sensor unit includes a light emitting element that emits light toward an object, two light receiving elements that receive light from the object, and a transmissive window that is disposed between the light emitting element and the object and transmits light emitted from the light emitting element. The two light receiving elements are disposed so as to be positioned on a circle concentric with the light emitting element and to be positioned in a range of less than 180 degrees around the light emitting element.

### CROSS-REFERENCE TO RELATED APPLICATION(S)

This application is based upon, and claims the benefit of priority to, Japanese Patent Application No. 2023-140837, filed on Aug. 31, 2023, the entire contents of which are herein incorporated by reference, and Japanese Patent Application No. 2023-141508, filed on Aug. 31, 2023, the entire contents of which are herein incorporated by reference.

### FIELD

Embodiments of the disclosure relate to a sensor unit and a toilet seat device.

### BACKGROUND

There is conventionally proposed a sensor that measures a blood flow state or the like by emitting light such as laser light toward a human body and receiving and analyzing scattered light reflected from the human body (see, for example, JP 5785267 B). Note that the sensor in the related art is configured to include a light emitting element that emits light and two light receiving elements that receive light.

When the above-described sensor is provided in a toilet seat, for example, a transmissive window that transmits the light emitted from the light emitting element may be provided between the sensor and the human body (user) to protect the sensor, for instance. However, when the transmissive window is provided, the light receiving elements may receive not only the scattered light reflected from the user but also reflected light that is reflected from the transmissive window. This reflected light is not light reflected from the user and thus may act as noise in a process of measuring the blood flow state. Additionally, reduction in the size of the sensor unit including the above-described sensor is desired due to storage space restrictions and design considerations.

### SUMMARY

A sensor unit includes a light emitting element that emits light toward an object, two light receiving elements that receive light from the object, and a transmissive window that is disposed between the light emitting element and the object and transmits light emitted from the light emitting element, wherein the two light receiving elements are disposed so as to be positioned on a circle concentric with the light emitting element and to be positioned in a range of less than 180 degrees around the light emitting element.

### BRIEF DESCRIPTION OF DRAWING(S)

FIG. 1 is a perspective view illustrating a flush toilet bowl device including a toilet seat device according to a first embodiment.

FIG. 2 is a block diagram illustrating a configuration example of a measurement system.

FIG. 3 is a cross-sectional view taken along line III-III in FIG. 1.

FIG. 4 is a side cross-sectional view illustrating a light emitting element, a light receiving element, and a transmissive window in a side view.

FIG. 5 is a side cross-sectional view illustrating the light emitting element, the light receiving element, and the transmissive window in a side view.

FIG. 6 is a top view illustrating the light emitting element and light receiving elements in a top view.

FIG. 7 is a top view illustrating a light emitting element and light receiving elements according to a modification of the first embodiment in a top view.

FIG. 8 is a side cross-sectional view illustrating a light emitting element, a light receiving element, and a transmissive window according to a second embodiment in a side view.

FIG. 9 is a side cross-sectional view illustrating the light emitting element, the light receiving element, and the transmissive window according to the second embodiment in a side view.

FIG. 10 is a top view illustrating the light emitting element and light receiving elements according to the second embodiment in a top view.

FIG. 11 is a cross-sectional view taken along line III-III in FIG. 1, illustrating a third embodiment.

FIG. 12 is a side cross-sectional view illustrating a light emitting element, a light receiving element, and a transmissive window in a side view.

FIG. 13 is a side cross-sectional view illustrating the light emitting element, the light receiving element, and the transmissive window in a side view.

FIG. 14 is a top view illustrating the light emitting element and light receiving elements in a top view.

FIG. 15 is a side cross-sectional view illustrating a light emitting element, a light receiving element, and a transmissive window according to a fourth embodiment in a side view.

FIG. 16 is a side cross-sectional view illustrating the light emitting element, the light receiving element, and the transmissive window according to the fourth embodiment in a side view.

FIG. 17 is a top view illustrating the light emitting element and light receiving elements according to the fourth embodiment in a top view.

### DESCRIPTION OF EMBODIMENT(S)

Embodiments of a sensor unit and a toilet seat device disclosed herein will be described below in detail with reference to the accompanying drawings. Note that the present invention is not limited to the embodiments described below.

### First Embodiment

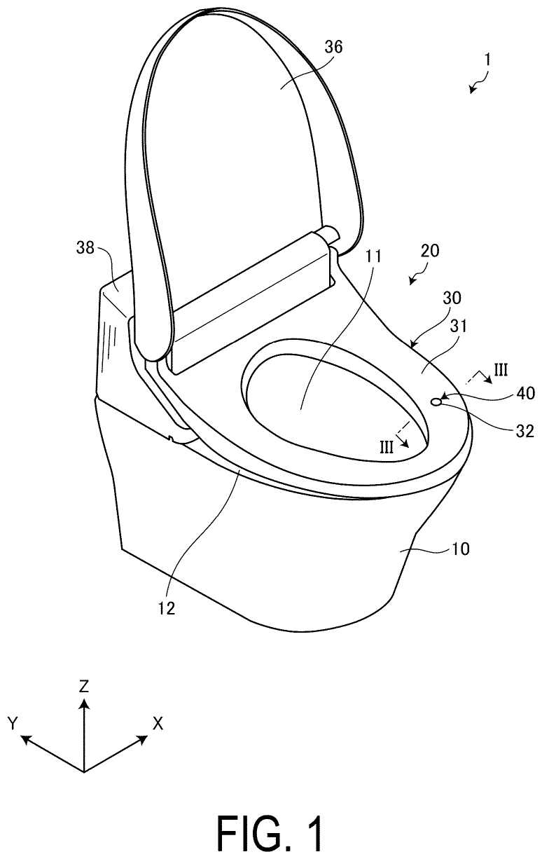

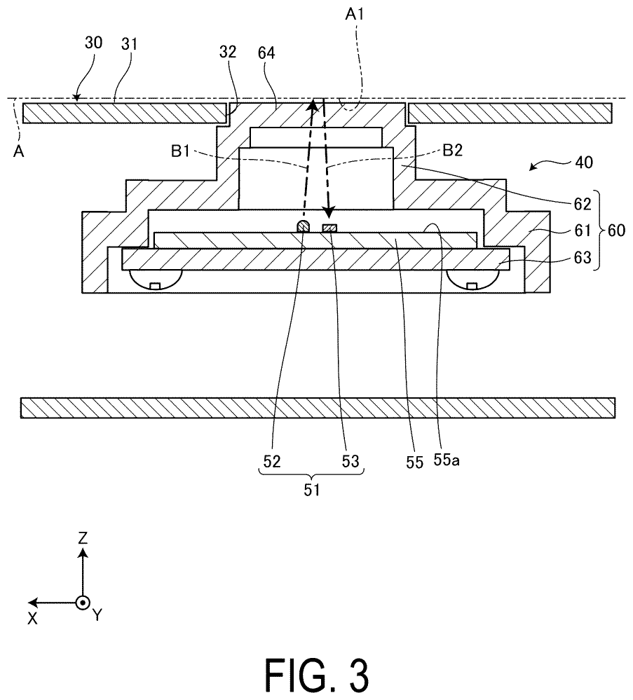

A toilet seat device including a sensor unit according to a first embodiment will be described with reference to FIG. 1. FIG. 1 is a perspective view illustrating a flush toilet bowl device including the toilet seat device according to the first embodiment. FIG. 1 and FIG. 3 and subsequent figures are all schematic views.

In FIG. 1 and other figures, to facilitate explanation, a three-dimensional orthogonal coordinate system is illustrated in which an X-axis direction, a Y-axis direction, and a Z-axis direction orthogonal to each other are defined, and a positive direction of the Z-axis is a vertically upward direction. In the following description, a positive X-axis direction may be referred to as “left”, a negative X-axis direction may be referred to as “right”, a positive Y-axis direction may be referred to as “rear”, and a negative Y-axis direction may be referred to as “front”. In other words, directions as viewed by a user sitting on a toilet seat 30 described later may be referred to as “left”, “right”, “rear”, and “front”. Accordingly, in the following description, the X-axis direction may be referred to as a left-right direction, the Y-axis direction may be referred to as a front-rear direction, and the Z-axis direction may be referred to as an up-down direction (vertical direction).

As illustrated in FIG. 1, a flush toilet bowl device 1 includes a flush toilet 10 and a toilet seat device 20. The flush toilet 10 includes a bowl 11. The bowl 11 is formed in a bowl shape that can receive waste. Flush water is released to flush the bowl 11. After the bowl 11 is flushed, the flush water is drained through a discharge conduit that is not illustrated. The flush toilet 10 is made of, for example, ceramic. Note that the flush toilet 10 is not limited to being made of ceramic and may be made of resin or a combination of ceramic and resin, for example.

The toilet seat device 20 is attached to an upper part of the flush toilet 10 and includes the toilet seat 30, a toilet lid 36, a functional unit 38, and a sensor unit 40. Note that the toilet seat device 20 may be detachably attached to the flush toilet 10 or may be attached to the flush toilet 10 in an integrated manner.

The toilet seat 30 is formed in an annular shape having an opening in the center, and is disposed on a rim 12 at a position at which the opening of the toilet seat 30 and the opening of the flush toilet 10 overlap. The toilet seat 30 is made of resin, for example, and includes a seat face 31 on which a user is seated. The seat face 31 supports the buttocks, thighs, and the like of the seated user. Further, the toilet seat 30 is formed to have a hollow shape, with the sensor unit 40 and the like provided inside the toilet seat 30.

The toilet lid 36 is attached to the toilet seat 30 in an openable and closable manner. The functional unit 38 contains, for example, a nozzle device and the like and jets washing water from the nozzle device toward the body of the user to wash the private part. Note that the toilet seat device 20 does not have to include the toilet lid 36 and the functional unit 38 as described above.

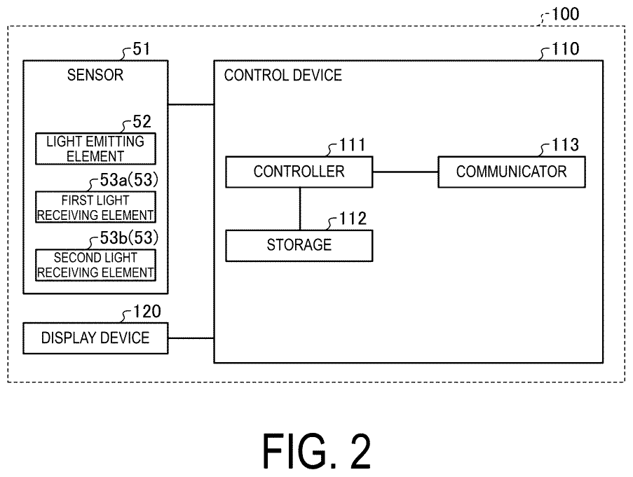

The sensor unit 40 includes a sensor 51 (see FIG. 2) that can measure various states related to the user using the flush toilet bowl device 1. For example, the sensor 51 is a sensor that can measure a blood flow state of the user seated on the toilet seat 30. As the sensor 51, an optical biosensor (e.g., a blood flow sensor that uses laser light) can be used. A measurement system including the sensor 51 will now be described with reference to FIG. 2. FIG. 2 is a block diagram illustrating a configuration example of the measurement system.

As illustrated in FIG. 2, a measurement system 100 includes the above-described sensor 51, a control device 110, and a display device 120. The sensor 51 includes a light emitting element 52 and two light receiving elements 53.

The light emitting element 52 emits, for example, laser light. Specifically, the light emitting element 52 emits laser light toward the user seated on the toilet seat 30, for example. More specifically, when the user is seated on the toilet seat 30, the light emitting element 52 emits laser light toward a measurement site of the buttocks or thigh of the user. Note that the light emitting element 52 may be configured to emit not only laser light but also other types of light, such as infrared rays and visible light. The user (to be precise, the measurement site of the user) to which light is emitted (which is irradiated with light) is an example of an object.

The two light receiving elements 53 receive light from the user (to be precise, the measurement site of the user). Note that one of the two light receiving elements 53 may be referred to as a “first light receiving element 53a”, and the other may be referred to as a “second light receiving element 53b”. The first and second light receiving elements 53a and 53b will be referred to as “the light receiving element 53” or “the two light receiving elements 53” when no particular distinction is needed.

More specifically, the light emitted from the light emitting element 52 (irradiation light, laser light) is reflected from a blood vessel (blood flow) of the user. Then, the two light receiving elements 53 receive the reflected light (scattered light). The sensor 51 outputs a signal related to the irradiation light or the scattered light, such as a frequency of the irradiation light or the scattered light, to the control device 110.

The control device 110 includes a controller 111, a storage 112, and a communicator 113. Note that the control device 110 is contained in, for example, the functional unit 38 (see FIG. 1) of the toilet seat device 20, but no limitation is intended.

The controller 111 is implemented by, for example, executing a program stored in the storage 112 by a central processing unit (CPU), a micro processing unit (MPU), or the like using a random access memory (RAM) as a working area. Note that it is possible to implement the controller 111 by hardware such as an application specific integrated circuit (ASIC) or a field programmable gate array (FPGA). The storage 112 includes a volatile memory and a nonvolatile memory and is implemented by, for example, a semiconductor memory element such as a RAM or a flash memory.

On the basis of the signal output from the sensor 51, the controller 111 measures a blood flow state, including, for example, a pulse rate, pulse rate fluctuations, and a blood flow rate, of the user. For example, the controller 111 performs a process of detecting a change in the flow rate of red blood cells flowing through a blood vessel using laser light. The controller 111 performs a process of detecting a frequency which is Doppler shifted due to red blood cells flowing through a blood vessel.

Specifically, the controller 111 amplifies (in other words, differentially amplifies) a difference between an electrical signal indicating the scattered light received by the first light receiving element 53a and an electrical signal indicating the scattered light received by the second light receiving element 53b. Note that this differential amplification and other processes may be performed by a differential amplifier circuit. Then, on the basis of the amplified signal, the controller 111 performs a process of detecting a change in the flow rate of red blood cells and a process of detecting a Doppler shifted frequency. That is, the controller 111 performs a process of analyzing the signals related to the irradiation light from the light emitting element 52 and the scattered light received by each of the two light receiving elements 53 to measure the blood flow state. Note that information indicating the measured blood flow state is not limited to a pulse rate, pulse rate fluctuations, and a blood flow amount of the user as described above, as long as it is information indicating a state related to the blood flow of the user, that is, biological information of the user.

The communicator 113 transmits and/or receives information to and/or from the display device 120. Note that the control device 110 and the display device 120 may be connected by wireless communication or may be connected by wired communication. The controller 111 outputs information indicating the measured blood flow state to the display device 120 via the communicator 113.

The display device 120 displays the information indicating the measured blood flow state. Thus, the user can confirm (understand) his/her own blood flow state. Note that the display device 120 may be a display in a remote controller that outputs an operation instruction for the toilet seat device 20 or a flushing instruction for the flush toilet 10, or a display in a mobile terminal used by a user. However, these are illustrative and no limitation is intended.

Note that the sensor 51 is not limited to the above-described sensor that measures the blood flow state of the user, and may include other types of sensors such as a load sensor that measures the weight of the user, and a body fat sensor that measures the body fat percentage of the user.

Referring back to FIG. 1, an opening 32 is formed in the seat face 31 of the toilet seat 30. The sensor unit 40 according to the embodiment is fixed to the toilet seat 30 such that a part of the sensor unit 40 is exposed through the opening 32.

When the above-described sensor 51 is provided in the toilet seat 30, a transmissive window 64 (see FIG. 3) that transmits light emitted from the light emitting element 52 is disposed between the sensor 51 and the user to protect the sensor 51 from contamination, impact, or the like. However, when the transmissive window 64 is disposed, the light receiving element 53 may receive not only the scattered light reflected from the user but also reflected light reflected from the transmissive window 64. Since this reflected light is not light reflected from the user and is not light indicating the blood flow state, the reflected light may act as noise in the process of measuring the blood flow state. Additionally, in the sensor unit 40 including the sensor 51, reduction in the size of the sensor unit 40 is desired due to storage space restrictions and design considerations.

Hence, in the present embodiment, a configuration is adopted in which the sensor unit 40 can be made smaller while achieving reduction of light received by the light receiving element 53 after being reflected from the transmissive window 64.

The sensor unit 40 and other elements according to the present embodiment will be described in detail with reference to FIG. 3. FIG. 3 is a cross-sectional view taken along line III-III in FIG. 1. Note that, in FIG. 3, the user A seated on the seat face 31 of the toilet seat 30 is indicated by a two dot chain line, and the measurement site of the user A is indicated by the reference sign “A1”.

As illustrated in FIG. 3, the sensor unit 40 includes the sensor 51, a sensor substrate 55, and a sensor case 60, and is fixed to the toilet seat 30.

As illustrated in FIG. 3, the sensor 51 is mounted on the sensor substrate 55. The sensor substrate 55 is made of resin, for example. The upper surface of the sensor substrate 55 is a main surface 55a. The sensor 51 is mounted near the center of the main surface 55a of the sensor substrate 55.

As described above, the sensor 51 includes the light emitting element 52 and the two light receiving elements 53 (one of them is not visible in FIG. 3). When the user A is seated on the toilet seat 30, the light emitting element 52 emits light (e.g., laser light) toward the measurement site A1 of the buttocks or thigh of the user A (see arrow B1). Note that the light emitting element 52 is also referred to as a light emitting unit or an irradiation unit.

The light receiving element 53 receives light from the user A (see arrow B2). Specifically, the light receiving element 53 receives scattered light that is light reflected from the measurement site A1 of the user A. Note that the light receiving element 53 is also referred to as a light receiving unit. Additionally, the sensor 51 may be one member in which the light emitting element 52 and the two light receiving elements 53 are integrated as one unit.

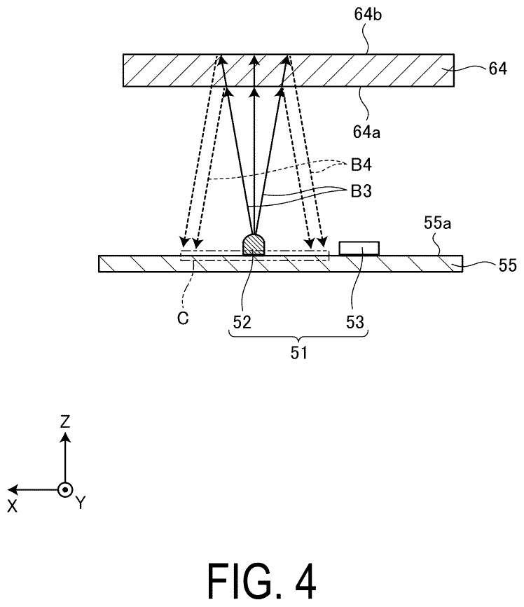

Note that the positions at which the light emitting element 52 and the two light receiving elements 53 are disposed, etc., will be described later with reference to FIGS. 4 to 6.

The sensor case 60 contains the sensor substrate 55 and other components. The sensor case 60 is made of resin, but no limitation is intended.

The sensor case 60 includes a main body 61, a protrusion 62, and a bottom surface 63. The main body 61 is formed in a hollow rectangular parallelepiped shape, for example. A lower surface side of the main body 61 is open.

The protrusion 62 is formed protruding upward from an upper part of the main body 61. The protrusion 62 is formed in a hollow cylindrical shape, for example. The transmissive window (sensor window) 64 is provided at an upper part of the protrusion 62.

The sensor case 60 is fixed to the toilet seat 30 in a state where the transmissive window 64 is exposed through the opening 32 of the toilet seat 30. Further, the light emitting element 52 and the light receiving elements 53 are disposed below the transmissive window 64. That is, the transmissive window 64 is disposed between the measurement site A1 of the user A, and the light emitting element 52 and light receiving elements 53. The transmissive window 64 transmits light emitted from the light emitting element 52 (see arrow B1). The transmissive window 64 also transmits scattered light reflected from the measurement site A1 of the user A (see arrow B2). In other words, the light emitting element 52 of the sensor 51 irradiates the user A with light via the transmissive window 64, and the light receiving elements 53 receive the scattered light reflected from the blood vessel of the user A via the transmissive window 64.

The bottom surface 63 is formed in a flat plate shape, for example. The bottom surface 63 is attached to the main body 61 from the opening side of the lower surface of the main body 61, and is fixed to the main body 61. Herein, the sensor substrate 55 is disposed and fixed between the main body 61 and the bottom surface 63.

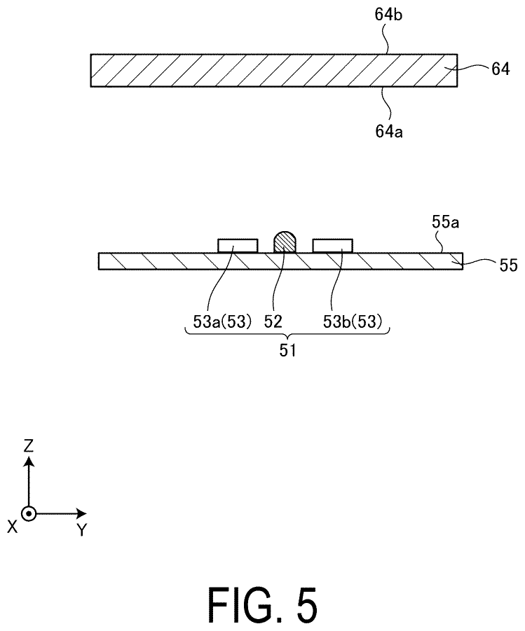

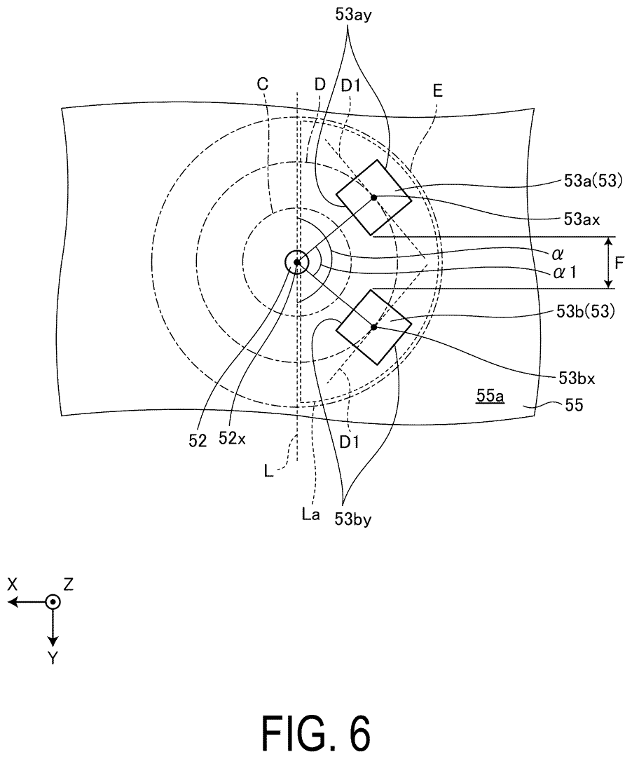

Next, configurations of the light emitting element 52, the two light receiving elements 53, and the transmissive window 64 according to the present embodiment will be described with reference to FIGS. 4 to 6. FIG. 4 is a side cross-sectional view illustrating the light emitting element 52, the light receiving element 53, and the transmissive window 64 when viewed from the side (specifically, a Y-axis direction view as viewed from the Y-axis direction). FIG. 5 is a side cross-sectional view illustrating the light emitting element 52, the light receiving element 53, and the transmissive window 64 when viewed from the side (specifically, an X-axis direction view as viewed from the X-axis direction). FIG. 6 is a top view (plan view) illustrating the light emitting element 52 and the light receiving elements 53 in a top view (plan view). Note that, in FIG. 6, illustration of the transmissive window 64 is omitted to facilitate understanding. Also in FIG. 6, a reference sign E indicates the range on the sensor substrate 55 on which the scattered light reflected from the blood vessel of the user A is incident.

As illustrated in FIGS. 4 to 6, the light emitting element 52 and the two light receiving elements 53 are disposed on the same plane. More specifically, the light emitting element 52 and the two light receiving elements 53 are disposed on the same main surface 55a of the sensor substrate 55.

As illustrated in FIG. 6, the two light receiving elements 53 are disposed so as to be positioned on a circle concentric with the light emitting element 52. Specifically, each of the two light receiving elements 53 is disposed such that a center portion of the light receiving element 53 is positioned on the circle concentric with the light emitting element 52. More specifically, the first light receiving element 53a is disposed such that a center portion 53ax is positioned on the circle concentric with the light emitting element 52, and the second light receiving element 53b is disposed such that a center portion 53bx is positioned on the circle concentric with the light emitting element 52. The circle concentric with the light emitting element 52 is a circle centered at a center portion 52x of the light emitting element 52, and an example of the concentric circle is indicated by reference sign D. As described above, since the two light receiving elements 53 are positioned on the circle D concentric with the light emitting element 52, the two light receiving elements 53 are each disposed at the same distance from the light emitting element 52.

The two light receiving elements 53 are disposed so as to be positioned in a range of less than 180 degrees around the light emitting element 52. In other words, the two light receiving elements 53 are disposed so as to be positioned in a range of less than 180 degrees in terms of a circumferential range around the light emitting element 52. To be more specific, in a top view, an angle of 180 degrees (a) around the light emitting element 52 is indicated by a virtual straight line L passing through the center portion 52x of the light emitting element 52, and the two light receiving elements 53 are disposed so as to be positioned in a range La of less than 180 degrees (a).

More specifically, in the range La of less than 180 degrees (a), the two light receiving elements 53 are disposed such that an angle al between a straight line connecting the first light receiving element 53a and the light emitting element 52 and a straight line connecting the second light receiving element 53b and the light emitting element 52 is, for example, from 60 degrees to 90 degrees. Specifically, in the range La of less than 180 degrees (a), the two light receiving elements 53 are disposed such that the angle α1 between a virtual straight line connecting the center portion 53ax of the first light receiving element 53a and the center portion 52x of the light emitting element 52 and a virtual straight line connecting the center portion 53bx of the second light receiving element 53b and the center portion 52x of the light emitting element 52 is, for example, from 60 degrees to 90 degrees. Note that, in the above description, the angle α1 is indicated by a specific numerical value, but this is merely an example and no limitation is intended, and it is possible to set the angle α1 to any value as long as the value is less than 180 degrees.

Note that, since the two light receiving elements 53 receive the scattered light reflected from the blood vessel of the user A, the two light receiving elements 53 are disposed within the range E on which the scattered light is incident.

Since the two light receiving elements 53 according to the present embodiment are disposed at the above-described positions, the sensor unit 40 can be made smaller while achieving reduction of light received by the light receiving element 53 after being reflected from the transmissive window 64.

That is, as illustrated in FIG. 4, light emitted from the light emitting element 52 (see arrow B3) is reflected from the transmissive window 64 (see arrow B4). The light reflected from the transmissive window 64 is incident on the light emitting element 52 and the vicinity of the light emitting element 52 on the sensor substrate 55. Note that, in FIGS. 4 and 6, a region in which the reflected light is incident on the sensor substrate 55 is indicated by reference sign C. Additionally, in FIG. 4, refraction of the light at the transmissive window 64 is omitted for simplification of illustration.

Since the two light receiving elements 53 are disposed so as to be positioned in a region outside a region C in which the reflected light is incident and which is concentric with the light emitting element 52, the light reflected from the transmissive window 64 is not incident on the light receiving elements 53 or is unlikely to be incident on the light receiving elements 53. In other words, it is possible to reduce light received by the light receiving elements 53 after being reflected from the transmissive window 64. Specifically, it is possible to reduce the reflected light that acts as noise in the process of measuring the blood flow state and is received by the light receiving elements 53, for example.

In addition, since the two light receiving elements 53 are disposed so as to be positioned on a circle concentric with the light emitting element 52, the scattered light can be uniformly received across the two light receiving elements 53. In other words, it is possible to suppress bias of the received scattered light. When differentially amplifying the signals indicating the scattered light received by the two light receiving elements 53, for example, the measurement accuracy of the blood flow state may deteriorate if the scattered light is biased. However, by disposing the two light receiving elements 53 as described above, it is possible to suppress deterioration of the measurement accuracy caused by bias of the scattered light.

In addition, since the two light receiving elements 53 are disposed so as to be positioned in the range La of less than 180 degrees around the light emitting element 52, the range in which the two light receiving elements 53 are disposed can be made relatively small, and thus the sensor unit 40 can be made smaller.

The two light receiving elements 53 are disposed spaced apart from each other. Specifically, the first light receiving element 53a and the second light receiving element 53b are disposed spaced apart from each other by a predetermined distance F. The predetermined distance F can be set to any value.

As described above, since the two light receiving elements 53 are disposed spaced apart from each other, the influence of reflected light (noise) can be suppressed to the extent possible. Specifically, if the transmissive window 64 is slanted due to, for example, angle variations or the like of the transmissive window 64, the reflected light may be slightly incident on the light receiving elements 53. Even in such a case, since the two light receiving elements 53 are disposed spaced apart from each other, it is possible to reduce the occurrence of the two light receiving elements 53 being affected by the reflected light at the same time, and thus reduce the influence of the reflected light (noise) to the extent possible.

As illustrated in FIG. 6, the two light receiving elements 53 are each formed to have a quadrangular shape in a top view. Additionally, the light receiving element 53 is disposed such that one side thereof is parallel to a tangent D1 to the circle D concentric with the light emitting element 52. Specifically, the first light receiving element 53a is disposed such that one side 53ay thereof is parallel to the tangent D1 to the circle D concentric with the light emitting element 52. Similarly, the second light receiving element 53b is disposed such that one side 53by thereof is parallel to the tangent D1 to the circle D concentric with the light emitting element 52. Note that the sides 53ay and 53by are, for example, at least one of a side facing the light emitting element 52 and a side opposite the side facing the light emitting element 52 among the four sides of the light receiving element 53.

With this configuration, since the light receiving element 53 is disposed such that one side thereof is parallel to the tangent D1 to the circle D concentric with the light emitting element 52, it is possible to reduce the occurrence of reflected light being incident on a corner of the light receiving element 53, for example. That is, it is possible to effectively reduce the reflected light received by the light receiving element 53.

As described above, the sensor unit 40 according to the first embodiment includes the light emitting element 52 that emits light toward the object (user A), the two light receiving elements 53 that each receive light from the object, and the transmissive window 64 that is disposed between the light emitting element 52 and the object and transmits the light from the light emitting element 52. The two light receiving elements 53 are disposed so as to be positioned on the circle D concentric with the light emitting element 52 and to be positioned in the range La of less than 180 degrees around the light emitting element 52. Thus, the sensor unit 40 can be made smaller while achieving reduction of light received by the light receiving element 53 after being reflected from the transmissive window 64.

### Modification of First Embodiment

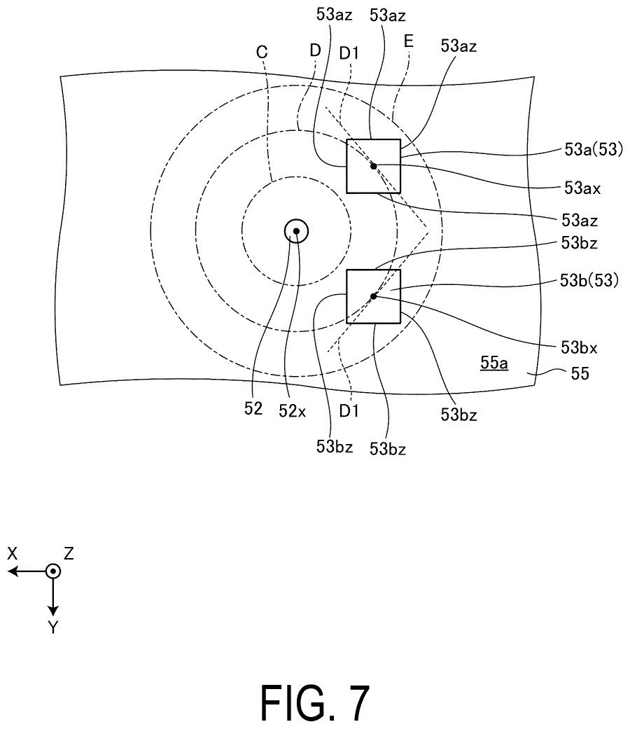

Next, a modification of the first embodiment will be described with reference to FIG. 7. FIG. 7 is a top view similar to FIG. 6, illustrating a light emitting element 52 and light receiving elements 53 according to the modification of the first embodiment in a top view. Note that, hereinafter, the same components as those of the first embodiment may be denoted by the same reference signs to omit repeated description.

As illustrated in FIG. 7, in the modification of the first embodiment, each light receiving element 53 having a quadrangular shape in a top view is disposed such that all four sides intersect with a tangent D1 to a circle D concentric with the light emitting element 52. More specifically, a first light receiving element 53a is disposed such that four sides 53az intersect with the tangent D1 to the circle D concentric with the light emitting element 52. Similarly, a second light receiving element 53b is disposed such that four sides 53bz intersect with the tangent D1 to the circle D concentric with the light emitting element 52.

As described above, even when the light receiving element 53 is disposed such that the four sides of the light receiving element 53 intersect with the tangent D1 to the circle D concentric with the light emitting element 52, it is possible to reduce the reflected light received by the light receiving elements 53, similar to the first embodiment.

### Second Embodiment

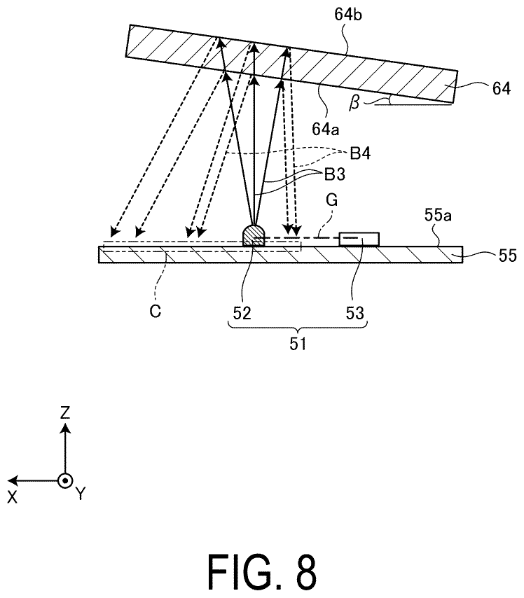

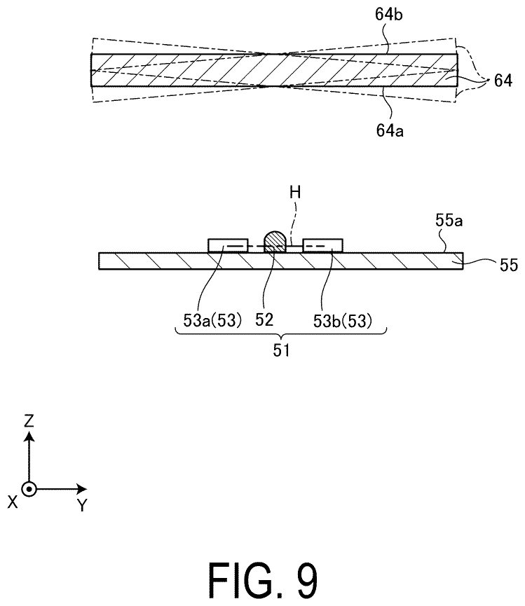

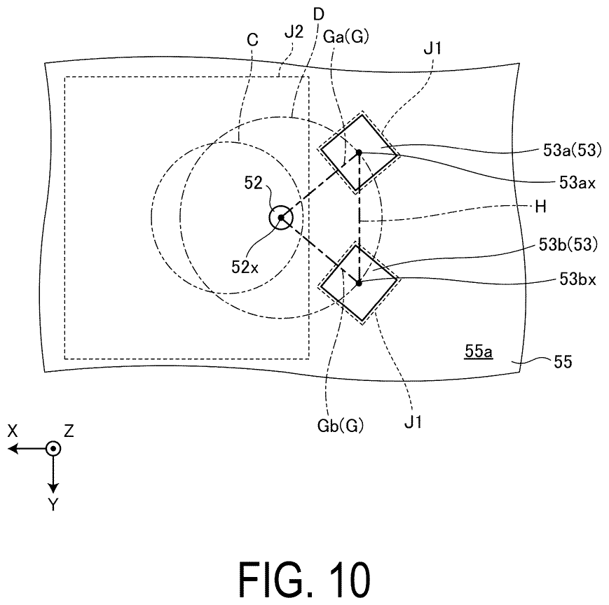

Next, a sensor unit 40 and other components according to a second embodiment will be described with reference to FIGS. 8 to 10. FIG. 8 is a side cross-sectional view illustrating a light emitting element 52, a light receiving element 53, and a transmissive window 64 when viewed from the side (specifically, a Y-axis direction view as viewed from the Y-axis direction). FIG. 9 is a side cross-sectional view illustrating the light emitting element 52, the light receiving element 53, and the transmissive window 64 when viewed from the side (specifically, an X-axis direction view as viewed from the X-axis direction). FIG. 10 is a top view illustrating the light emitting element 52 and light receiving elements 53 in a top view.

Note that, to facilitate understanding, the following description may refer to straight lines G and H which virtually connect the light emitting element 52 and the two light receiving elements 53, as illustrated in FIG. 10. Specifically, in a top view, a straight line connecting a center portion 52x of the light emitting element 52 and a center portion 53ax of a first light receiving element 53a is referred to as a “first straight line Ga”. Further, in a top view, a straight line connecting the center portion 52x of the light emitting element 52 and a center portion 53bx of a second light receiving element 53b is referred to as a “second straight line Gb”. In addition, a straight line connecting the two light receiving elements 53 in a top view, in other words, a straight line connecting the center portion 53ax of the first light receiving element 53a and the center portion 53bx of the second light receiving element 53b is referred to as an “inter-light-receiving-element straight line H”.

As illustrated in FIG. 10, the light emitting element 52 and the two light receiving elements 53 are disposed such that one of the straight lines G connecting the light emitting element 52 and one of the two light receiving elements 53 and the other straight line G connecting the light emitting element 52 and the other of the two light receiving elements 53 and the inter-light-receiving-element straight line H form a triangular shape in a top view. For example, the light emitting element 52 and the two light receiving elements 53 are disposed to form an isosceles-triangular shape in which the first straight line Ga and the second straight line Gb have the same length. That is, the two light receiving elements 53 are disposed so as to be positioned on a circle D concentric with the light emitting element 52.

In the second embodiment, with respect to the light emitting element 52 and the two light receiving elements 53 disposed as described above, the transmissive window 64 is provided to be slanted so that light reflected from the transmissive window 64 is not incident on the light receiving elements 53.

Specifically, as illustrated in FIG. 8, the transmissive window 64 is provided to be slanted with respect to one of the straight lines G connecting the light emitting element 52 and one of the two light receiving elements and the other straight line G connecting the light emitting element 52 and the other of the two light receiving elements. More specifically, the transmissive window 64 is provided to be slanted at a slant angle β in a side view with respect to one of the straight lines G (to be precise, the first straight line Ga and the second straight line Gb (see FIG. 10)) connecting the light emitting element 52 and one of the two light receiving elements and the other straight line G connecting the light emitting element 52 and the other of the two light receiving elements. In other words, the transmissive window 64 is provided to be slanted at the slant angle β in the side view with respect to the main surface 55a of a sensor substrate 55 including the first straight line Ga and the second straight line Gb.

That is, the transmissive window 64 includes a lower window surface 64a facing the light emitting element 52 and an upper window surface 64b on a side opposite to the window surface 64a, and the window surfaces 64a and 64b are slanted with respect to the straight line G.

Since the transmissive window 64 according to the second embodiment is provided to be slanted as described above, it is possible to further reduce light received by the light receiving elements 53 after being reflected from the transmissive window 64. That is, since the transmissive window 64 is provided to be slanted as described above, the light reflected from the transmissive window 64 is incident on the sensor substrate 55 at positions other than those of the light receiving elements 53. In other words, the light reflected from the transmissive window 64 is not incident on or hardly incident on the light receiving elements 53 because the transmissive window 64 is slanted.

As described above, in the present embodiment, the transmissive window 64 is provided to be slanted with respect to one of the straight lines G (the first straight line Ga and the second straight line Gb) connecting the light emitting element 52 and one of the two light receiving elements 53 and the other straight line G connecting the light emitting element 52 and the other of the two light receiving elements 53. With this configuration, it is possible to reduce light received by the light receiving elements 53 after being reflected from the transmissive window 64. Specifically, it is possible to reduce the reflected light that acts as noise in the process of measuring a blood flow state and is received by the light receiving elements 53, for example.

As illustrated in FIG. 10, the transmissive window 64 is slanted such that the light reflected from the transmissive window 64 (see region C) is incident on a region J2, which is a region other than regions J1 where the two light receiving elements 53 are disposed. With this configuration, since the transmissive window 64 is slanted, it is possible to prevent the light reflected from the transmissive window 64 from being incident on the light receiving elements 53 and hence, it is possible to further reduce the reflected light received by the light receiving elements 53.

As illustrated in FIG. 9, the transmissive window 64 is provided such that the window surfaces 64a and 64b are parallel to the inter-light-receiving-element straight line H connecting the two light receiving elements 53. Specifically, the transmissive window 64 is provided such that both the lower window surface 64a and the upper window surface 64b are parallel to the inter-light-receiving-element straight line H.

Accordingly, it is possible to reduce the occurrence of the reflected light being biased toward the first light receiving element 53a or the second light receiving element 53b. That is, when the window surfaces 64a and 64b of the transmissive window 64 are slanted with respect to the inter-light-receiving-element straight line H, the reflected light is biased toward either the first light receiving element 53a or the second light receiving element 53b, and the amount of incident reflected light (noise) differs between the two light receiving elements 53, which may cause the measurement accuracy to deteriorate. By providing the transmissive window 64 such that the window surfaces 64a and 64b are parallel to the inter-light-receiving-element straight line H connecting the two light receiving elements 53, even if the reflected light is slightly incident on the light receiving elements 53, for example, the amount of incident reflected light (in other words, noise) can be made uniform across the two light receiving elements 53. Therefore, at the time of differential amplification of the signals indicating the light received by the two light receiving elements 53, for example, the influence of the reflected light (noise) can be reduced to the extent possible. As a result, deterioration of the measurement accuracy can be suppressed.

Note that, in the above description, the transmissive window 64 is provided such that the window surfaces 64a and 64b are parallel to the inter-light-receiving-element straight line H, but no limitation is intended. That is, as indicated by imaginary lines in FIG. 9, the transmissive window 64 may be provided to be slanted such that the window surfaces 64a and 64b are not parallel to the inter-light-receiving-element straight line H.

### Third Embodiment

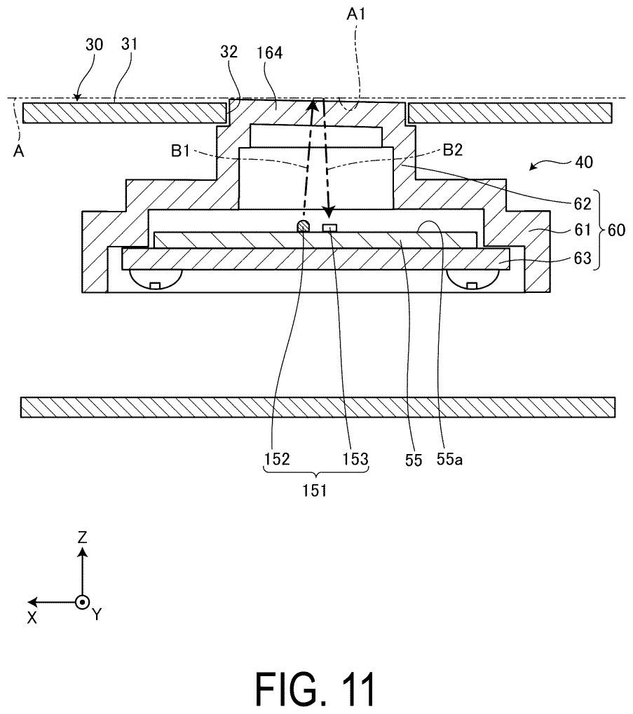

Next, a sensor unit 40 and other components according to a third embodiment will be described with reference to FIGS. 11 to 14. In the third embodiment and a fourth embodiment to be described later, some configurations corresponding to the configurations of the first and/or second embodiment may be denoted by adding “1” to the head of the reference sign. That is, the sensor 51, the light emitting element 52, the light receiving element 53, and the transmissive window 64 may be referred to as a “sensor 151”, a “light emitting element 152”, a “light receiving element 153”, and a “transmissive window 164”, respectively.

As described above, the transmissive window 164 (see FIG. 11) that transmits light from the light emitting element 152 is disposed between the sensor 151 and a user. However, the light receiving element 153 may receive not only scattered light reflected from the user but also reflected light reflected from the transmissive window 164 depending on arrangement and an angle of the transmissive window 164 relative to the sensor 151. Since this reflected light is not light reflected from the user and is not light indicating a blood flow state, the reflected light may act as noise in a process of measuring the blood flow state.

Hence, in the third embodiment, a configuration is adopted in which it is possible to reduce light received by the light receiving element 153 after being reflected from the transmissive window 164.

The sensor unit 40 and other components according to the third embodiment will be described in detail with reference to FIG. 11. FIG. 11 is a cross-sectional view taken along line III-III in FIG. 1, illustrating the third embodiment.

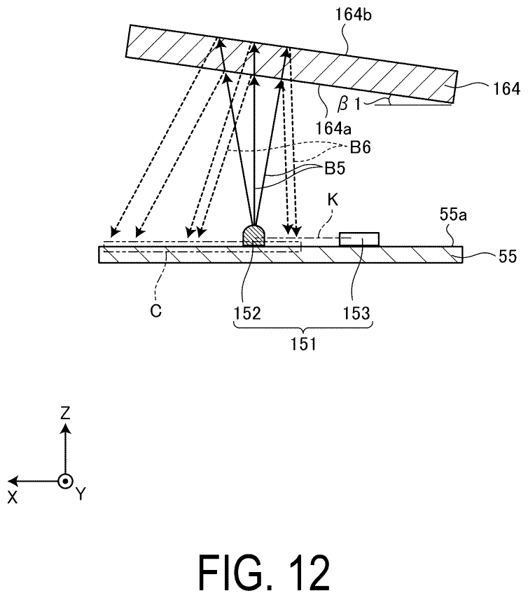

As illustrated in FIG. 11, the sensor unit 40 according to the third embodiment includes the sensor 151, a sensor substrate 55, and a sensor case 60, and is fixed to a toilet seat 30. As described above, the sensor 151 includes the light emitting element 152 and two light receiving elements 153 (one of them is not visible in FIG. 11). Note that the positions at which the light emitting element 152 and the two light receiving elements 153 are disposed, etc., will be described below with reference to FIGS. 12 to 14. Further, a detailed configuration of the transmissive window 164 of the sensor case 60 will also be described below with reference to FIGS. 12 to 14.

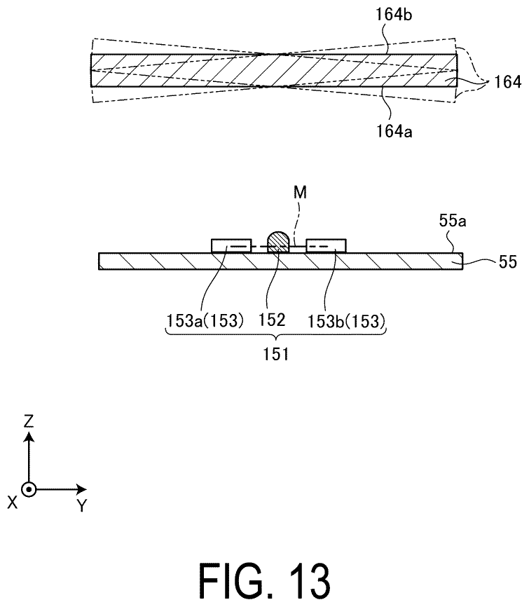

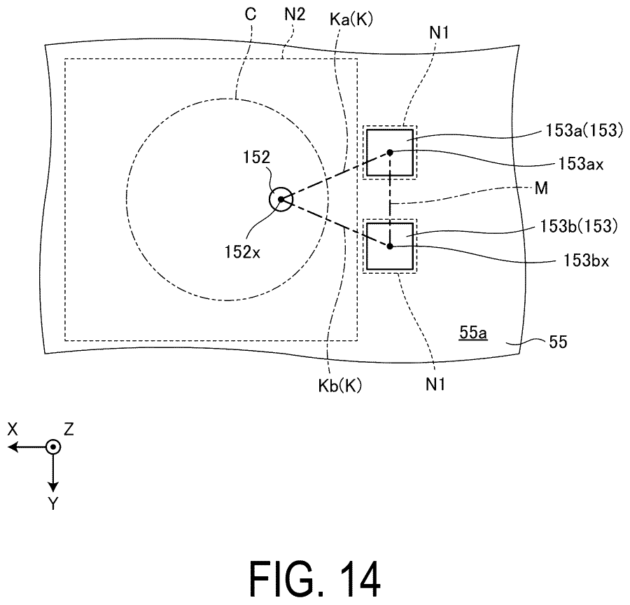

Next, configurations of the light emitting element 152, the two light receiving elements 153, and the transmissive window 164 according to the third embodiment will be described with reference to FIGS. 12 to 14. FIG. 12 is a side cross-sectional view illustrating the light emitting element 152, the light receiving element 153, and the transmissive window 164 when viewed from the side (specifically, a Y-axis direction view as viewed from the Y-axis direction). FIG. 13 is a side cross-sectional view illustrating the light emitting element 152, the light receiving element 153, and the transmissive window 164 when viewed from the side (specifically, an X-axis direction view as viewed from the X-axis direction). FIG. 14 is a top view (plan view) illustrating the light emitting element 152 and the light receiving elements 153 in a top view (plan view). Note that, in FIG. 14, illustration of the transmissive window 164 is omitted to facilitate understanding.

As illustrated in FIGS. 12 to 14, the light emitting element 152 and the two light receiving elements 153 are disposed on the same plane. More specifically, the light emitting element 152 and the two light receiving elements 153 are disposed on the same main surface 55a of the sensor substrate 55.

Note that, to facilitate understanding, the following description may refer to straight lines K and M which virtually connect the light emitting element 152 and the two light receiving elements 153, as illustrated in FIG. 14. Specifically, in a top view, a straight line connecting a center portion 152x of the light emitting element 152 and a center portion 153ax of a first light receiving element 153a is referred to as a “first straight line Ka”. Further, in a top view, a straight line connecting the center portion 152x of the light emitting element 152 and a center portion 153bx of a second light receiving element 153b is referred to as a “second straight line Kb”. In addition, a straight line connecting the two light emitting elements 152 in a top view, in other words, a straight line connecting the center portion 153ax of the first light receiving element 153a and the center portion 153bx of the second light receiving element 153b is referred to as the “inter-light-receiving-element straight line M”.

As illustrated in FIG. 14, the light emitting element 152 and the two light receiving elements 153 are disposed such that one of the straight lines K connecting the light emitting element 152 and one of the two light receiving elements 153, the other straight line K connecting the light emitting element 152 and the other of the two light receiving elements 153, and the inter-light-receiving-element straight line M form a triangular shape in a top view. For example, the light emitting element 152 and the two light receiving elements 153 are disposed to form an isosceles-triangular shape in which the first straight line Ka and the second straight line Kb have the same length.

The transmissive window 164 according to the third embodiment is provided to be slanted with respect to the light emitting element 152 and the two light receiving elements 153 disposed as described above such that the light reflected from the transmissive window 164 is not incident on the light receiving element 153.

As illustrated in FIG. 12, the transmissive window 164 is provided to be slanted with respect to one of the straight lines K connecting the light emitting element 152 and one of the two light receiving elements and the other straight line K connecting the light emitting element 152 and the other of the two light receiving elements. Specifically, the transmissive window 164 is provided to be slanted at a slant angle β1 in a side view with respect to one of the straight lines K (to be precise, the first straight line Ka and the second straight line Kb (see FIG. 14)) connecting the light emitting element 152 and one of the two light receiving elements and the other straight line K connecting the light emitting element 152 and the other of the two light receiving elements. In other words, the transmissive window 164 is provided to be slanted at the slant angle β1 in a side view with respect to the main surface 55a of the sensor substrate 55 including the first straight line Ka and the second straight line Kb.

In other words, the transmissive window 164 includes a lower window surface 164a facing the light emitting element 152 and an upper window surface 164b on a side opposite to the window surface 164a, and the window surfaces 164a and 164b are provided to be slanted with respect to the straight line K.

Since the transmissive window 164 according to the third embodiment is provided to be slanted as described above, it is possible to reduce light received by the light receiving elements 153 after being reflected from the transmissive window 164. That is, as illustrated in FIG. 12, light emitted from the light emitting element 152 (see arrow B5) is reflected from the transmissive window 164 (see arrow B6). Since the transmissive window 164 is provided to be slanted as described above, the light reflected from the transmissive window 164 is incident on the sensor substrate 55 at positions other than those of the light receiving elements 153. In other words, the light reflected from the transmissive window 164 is not incident on or hardly incident on the light receiving elements 153 because the transmissive window 164 is slanted. Note that, in FIGS. 12 and 14, a region in which the reflected light is incident on the sensor substrate 55 is indicated by reference sign C. Additionally, in FIG. 12, refraction of the light at the transmissive window 164 is omitted for simplification of illustration.

As described above, in the third embodiment, the transmissive window 164 is provided to be slanted with respect to one of the straight lines K (the first straight line Ka and the second straight line Kb) connecting the light emitting element 152 and one of the two light receiving elements 153 and the other straight line K connecting the light emitting element 152 and the other of the two light receiving elements 153. Accordingly, it is possible to reduce light received by the light receiving elements 153 after being reflected from the transmissive window 164. Specifically, it is possible to reduce the reflected light that acts as noise in the process of measuring the blood flow state and is received by the light receiving elements 153, for example.

As illustrated in FIG. 14, the transmissive window 164 is slanted such that light reflected from the transmissive window 164 (see region C) is incident on a region N2, which is a region other than regions N1 where the two light receiving elements 153 are disposed. With this configuration, it is possible to prevent the light reflected from the transmissive window 164 from being incident on the light receiving elements 153, due to a slant of the transmissive window 164, and hence, it is possible to further reduce the reflected light received by the light receiving elements 153.

As illustrated in FIG. 14, the two light receiving elements 153 are each disposed at the same distance from the light emitting element 152. Specifically, the two light receiving elements 153 are disposed such that the first straight line Ka and the second straight line Kb have the same length (Ka=Kb).

With this configuration, even if the reflected light is slightly incident on the light receiving elements 153, for example, the amount of incident reflected light (in other words, noise) can be made uniform across the two light receiving elements 153. Therefore, at the time of differential amplification of the signals indicating the light received by the two light receiving elements 153, for example, the influence of the reflected light (noise) can be reduced to the extent possible.

Further, as illustrated in FIG. 13, the transmissive window 164 is provided such that the window surfaces 164a and 164b are parallel to the inter-light-receiving-element straight line M connecting the two light-receiving elements 153. More specifically, the transmissive window 164 is provided such that both the lower window surface 164a and the upper window surface 164b are parallel to the inter-light-receiving-element straight line M.

Accordingly, it is possible to reduce the occurrence of the reflected light being biased toward the first light receiving element 153a or the second light receiving element 153b. That is, when the window surfaces 164a and 164b of the transmissive window 164 are slanted with respect to the inter-light-receiving-element straight line M, the reflected light is biased toward either the first light receiving element 153a or the second light receiving element 153b, and the amount of incident reflected light (noise) differs between the two light receiving elements 153, which may cause the measurement accuracy to deteriorate. By providing the transmissive window 164 such that the window surfaces 164a and 164b are parallel to the inter-light-receiving-element straight line M connecting the two light receiving elements 153, even if the reflected light is slightly incident on the light receiving elements 153, for example, the amount of incident reflected light (in other words, noise) can be made uniform across the two light receiving elements 153. Therefore, at the time of differential amplification of the signals indicating the light received by the two light receiving elements 153, for example, the influence of the reflected light (noise) can be reduced to the extent possible. As a result, deterioration of the measurement accuracy can be suppressed.

Note that, in the above description, the transmissive window 164 is provided such that the window surfaces 164a and 164b are parallel to the inter-light-receiving-element straight line M, but no limitation is intended. That is, as indicated by imaginary lines in FIG. 13, the transmissive window 164 may be provided to be slanted such that the window surfaces 164a and 164b are not parallel to the inter-light-receiving-element straight line M.

As described above, the sensor unit 40 according to the third embodiment includes the light emitting element 152 that emits light toward an object (user A), the two light receiving elements 153 that each receive light from the object, and the transmissive window 164 that is disposed between the light emitting element 152 and the object and transmits the light from the light emitting element 152. The light emitting element 152 and the two light receiving elements 153 are disposed on the same plane. The transmissive window 164 is provided to be slanted with respect to one of the straight lines K connecting the light emitting element 152 and one of the two light receiving elements 153 and the other straight line K connecting the light emitting element 152 and the other of the two light receiving elements 153. With this configuration, it is possible to reduce light received by the light receiving elements 153 after being reflected from the transmissive window 164.

### Fourth Embodiment

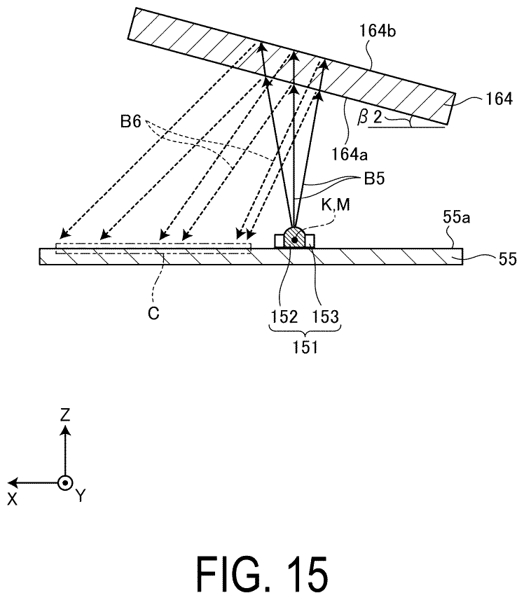

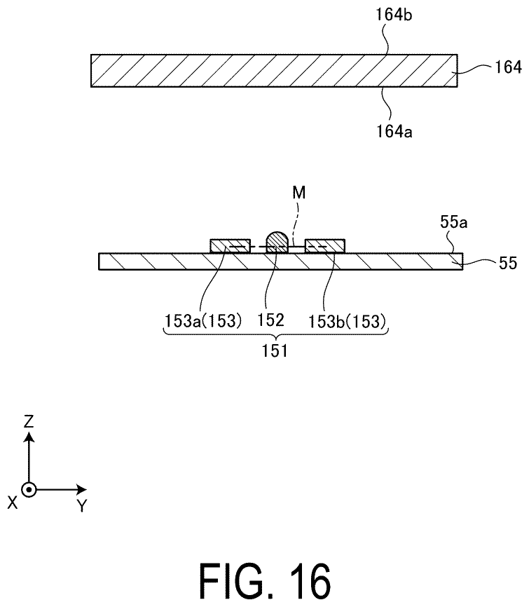

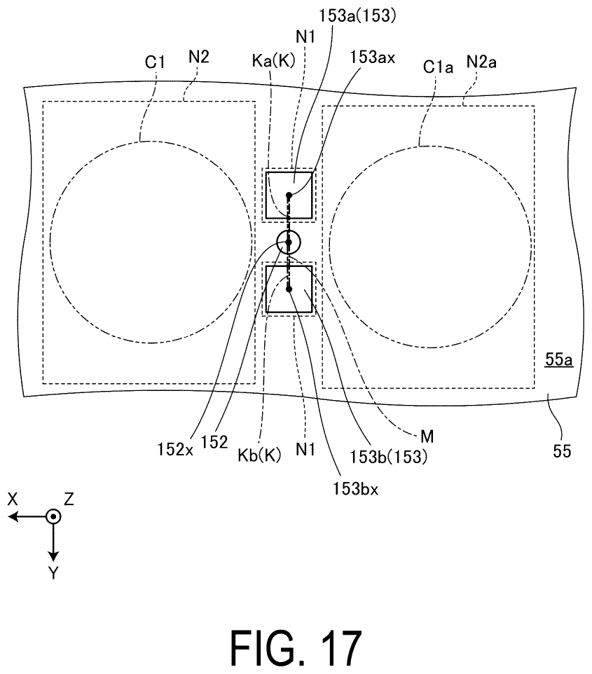

Next, a sensor unit 40 and other components according to a fourth embodiment will be described with reference to FIGS. 15 to 17. FIG. 15 is a side cross-sectional view illustrating a light emitting element 152, a light receiving element 153, and a transmissive window 164 when viewed from the side (specifically, a Y-axis direction view as viewed from the Y-axis direction). FIG. 16 is a side cross-sectional view illustrating the light emitting element 152, the light receiving element 153, and the transmissive window 164 when viewed from the side (specifically, an X-axis direction view as viewed from the X-axis direction). FIG. 17 is a top view illustrating the light emitting element 152 and light receiving elements 153 in a top view. Note that, in FIG. 17, illustration of the transmissive window 164 is omitted to facilitate understanding. Note that, hereinafter, the same components as those of the third embodiment may be denoted by the same reference signs to omit repeated description.

As illustrated in FIGS. 15 to 17, the light emitting element 152 and the two light receiving elements 153 are disposed such that one of the straight lines K connecting the light emitting element 152 and one of the two light receiving elements 153, the other straight line K connecting the light emitting element 152 and the other of the two light receiving elements 153, and an inter-light-receiving-element straight line M are parallel to each other in a top view, in other words, are disposed on the same straight line.

As illustrated in FIG. 15, the transmissive window 164 according to the fourth embodiment is provided to be slanted with respect to the one of straight lines K connecting the light emitting element 152 and one of the two light receiving elements, the other straight line K connecting the light emitting element 152 and the other of the two light receiving elements, and the inter-light-receiving-element straight line M. Specifically, the transmissive window 164 is provided to be slanted at a slant angle β2 in a side view with respect to one of the straight lines K (to be precise, a first straight line Ka and a second straight line Kb (see FIG. 17)) connecting the light emitting element 152 and one of the two light receiving elements, the other straight line K connecting the light emitting element 152 and the other of the two light receiving elements, and the inter-light-receiving-element straight line M. In other words, the transmissive window 164 is provided to be slanted at the slant angle β2 in a side view with respect to the main surface 55a of a sensor substrate 55 including the first straight line Ka, the second straight line Kb, and the inter-light-receiving-element straight line M.

Since the transmissive window 164 according to the fourth embodiment is provided to be slanted as described above, similar to the third embodiment, it is possible to reduce light received by the light receiving elements 153 after being reflected from the transmissive window 164. That is, as illustrated in FIG. 15, light emitted from light emitting element 152 (see arrow B5) is reflected from the transmissive window 164 (see arrow B6). Since the transmissive window 164 is provided to be slanted as described above, the light reflected from the transmissive window 164 is incident on the sensor substrate 55 at positions other than those of the light receiving elements 153. In other words, the light reflected from the transmissive window 164 is not incident on or hardly incident on the light receiving elements 153 because the transmissive window 164 is slanted.

As described above, in the fourth embodiment, the transmissive window 164 is provided to be slanted with respect to one of the straight lines K (the first straight line Ka and the second straight line Kb) connecting the light emitting element 152 and one of the two light receiving elements 153, the other straight line K connecting the light emitting element 152 and the other of the two light receiving elements 153, and the inter-light-receiving-element straight line M. With this configuration, it is possible to reduce light received by the light receiving elements 153 after being reflected from the transmissive window 164. Specifically, it is possible to reduce the reflected light that acts as noise in a process of measuring a blood flow state and is received by the light receiving elements 153.

As illustrated in FIG. 17, the transmissive window 164 is slanted such that light reflected from the transmissive window 164 (see region C1) is incident on a region N2, which is a region other than regions N1 where the two light receiving elements 153 are disposed. With this configuration, it is possible to prevent the light reflected from the transmissive window 164 from being incident on the light receiving elements 153, due to a slant of the transmissive window 164, and hence, it is possible to further reduce the reflected light received by the light receiving elements 153.

Note that, in the fourth embodiment described above, the reflected light (see region C1) is incident on the region N2 on the X-axis positive direction side with respect to the light receiving elements 153, but no limitation is intended. That is, the transmissive window 164 may be slanted such that the reflected light (see region C1a) is incident on a region N2a on the X-axis negative direction side with respect to the light receiving element 153.

Note that the above-described embodiments and modifications can be combined as appropriate. For example, when there are a plurality of sensor units 40, the first embodiment and the second embodiment may be combined, or the third embodiment and the fourth embodiment may be combined.

An object of an aspect of an embodiment is to provide a sensor unit and a toilet seat device that can be made smaller while achieving reduction of light received by a light receiving element after being reflected from a transmissive window.

A sensor unit according to an aspect of an embodiment includes a light emitting element that emits light toward an object, two light receiving elements that receive light from the object, and a transmissive window that is disposed between the light emitting element and the object and that transmits light emitted from the light emitting element. The two light receiving elements are disposed so as to be positioned on a circle concentric with the light emitting element and to be positioned in a range of less than 180 degrees around the light emitting element.

With this configuration, since the two light receiving elements are disposed so as to be positioned on a circle concentric with the light emitting element, scattered light can be uniformly received across the two light receiving elements. In other words, it is possible to suppress bias of the received scattered light. When differentially amplifying signals indicating the scattered light received by the two light receiving elements, for example, the measurement accuracy of the blood flow state may deteriorate if the scattered light is biased. However, by disposing the two light receiving elements as described above, it is possible to suppress deterioration of the measurement accuracy caused by bias of the scattered light.

In addition, since the two light receiving elements are disposed so as to be positioned in the range of less than 180 degrees around the light emitting element, the range in which the two light receiving elements are disposed can be made relatively small, and thus the sensor unit can be made smaller.

Further, the two light receiving elements are disposed spaced apart from each other.

Since the two light receiving elements are disposed spaced apart from each other, the influence of the reflected light (noise) can be reduced to the extent possible. Specifically, if the transmissive window is slanted due to angle variations or the like of the transmissive window, the reflected light may be slightly incident on the light receiving elements. Even in such a case, since the two light receiving elements are disposed spaced apart from each other, it is possible to reduce the occurrence of the two light receiving elements being affected by the reflected light at the same time, and thus reduce the influence of the reflected light (noise) to the extent possible.

Further, the two light receiving elements are each formed in a quadrangular shape in a top view, and are disposed such that one side of each light receiving element is parallel to a tangent to the circle concentric with the light emitting element.

With this configuration, since the light receiving elements are disposed such that one side of each light receiving element is parallel to a tangent to the circle concentric with the light emitting element, it is possible to reduce the occurrence of reflected light being incident on a corner of the light receiving element, for example. That is, it is possible to effectively reduce the reflected light received by the light receiving element.

Further, the light emitting element and the two light receiving elements are disposed on the same plane, and the transmissive window is provided to be slanted with respect to a straight line connecting the light emitting element and one of the two light receiving elements and another straight line connecting the light emitting element and the other of the two light receiving elements.

With this configuration, it is possible to reduce light received by the light receiving elements after being reflected from the transmissive window. Specifically, the light emitted from the light emitting element is reflected from the transmissive window. Since the transmissive window is provided to be slanted as described above, the light reflected from the transmissive window is incident on a position other than those of the light receiving elements on a sensor substrate, for example. In other words, the light reflected from the transmissive window is not incident on or hardly incident on the light receiving elements because the transmissive window is slanted. With this configuration, it is possible to reduce light received by the light receiving elements after being reflected from the transmissive window. Specifically, it is possible to reduce the reflected light that acts as noise in a process of measuring a blood flow state and is received by the light receiving elements for example.

Further, each of the two light receiving elements is disposed such that a center portion of each light receiving element is positioned on the circle concentric with the light emitting element.

With this configuration, since each of the two light receiving elements is disposed such that the center portion of each light receiving element is positioned on the circle concentric with the light emitting element, scattered light can be uniformly received across the two light receiving elements. In other words, it is possible to suppress bias of the received scattered light. When differentially amplifying signals indicating the scattered light received by the two light receiving elements, for example, the measurement accuracy of the blood flow state may deteriorate if the scattered light is biased. However, by disposing the two light receiving elements as described above, it is possible to suppress deterioration of the measurement accuracy caused by bias of the scattered light.

Each of the two light receiving elements is disposed at the same distance from the light emitting element.

With this configuration, even if the reflected light is slightly incident on the light receiving elements, for example, the amount of incident reflected light (in other words, noise) can be made uniform across the two light receiving elements. Therefore, at the time of differential amplification of the signals indicating the light received by the two light receiving elements, for example, the influence of the reflected light (noise) can be reduced to the extent possible.

Further, the transmissive window is provided such that a window surface of the transmissive window is parallel to a straight line connecting the two light receiving elements.

With this configuration, it is possible to reduce the occurrence of the reflected light being biased toward one of the two light receiving elements. That is, when the window surface of the transmissive window is slanted with respect to the straight line connecting the light receiving elements, the reflected light is biased toward one of the two light receiving elements, and the amount of incident reflected light (noise) differs between the two light receiving elements, which may cause the measurement accuracy to deteriorate. By providing the transmissive window such that the window surface is parallel to the straight line connecting the two light receiving elements, even if the reflected light is slightly incident on the light receiving elements, for example, the amount of incident reflected light (in other words, noise) can be made uniform across the two light receiving elements. Therefore, at the time of differential amplification of the signals indicating the light received by the two light receiving elements, for example, the influence of the reflected light (noise) can be reduced to the extent possible. As a result, deterioration of the measurement accuracy can be suppressed.

Further, the transmissive window is slanted such that reflected light emitted from the light emitting element and reflected from the transmissive window is incident on a region other than a region where the two light receiving elements are disposed.

With this configuration, it is possible to prevent the light reflected from the transmissive window from being incident on the light receiving elements, due to a slant of the transmissive window and it is possible to further reduce the reflected light received by the light receiving elements.

Further, a toilet seat device includes a sensor unit and a toilet seat provided with the sensor unit. The sensor unit includes a light emitting element that emits light toward an object, two light receiving elements that receive light from the object, and a transmissive window that is disposed between the light emitting element and the object and transmits light emitted from the light emitting element. The two light receiving elements are disposed so as to be positioned on a circle concentric with the light emitting element and to be positioned in a range of less than 180 degrees around the light emitting element.

Accordingly, the sensor unit provided in the toilet seat can be made smaller while achieving reduction of light received by the light receiving element after being reflected from the transmissive window.

Further, in the toilet seat device, the light emitting element and the two light receiving elements are disposed on the same plane, and the transmissive window is provided to be slanted with respect to a straight line connecting the light emitting element and one of the two light receiving elements and another straight line connecting the light emitting element and the other of the two light receiving elements.

With this configuration, in the sensor unit provided in the toilet seat, it is possible to reduce light received by the light receiving element after being reflected from the transmissive window.

According to an aspect of an embodiment, the sensor unit can be made smaller while achieving reduction of light received by the light receiving element after being reflected from the transmissive window.

### Supplementary Note

(1)

A sensor unit including:
a light emitting element that emits light toward an object;
two light receiving elements that receive light from the object; and
a transmissive window that is disposed between the light emitting element and the object and transmits light emitted from the light emitting element, in which
the two light receiving elements are disposed so as to be positioned on a circle concentric with the light emitting element and to be positioned in a range of less than 180 degrees around the light emitting element.
   (2)

The sensor unit according to (1), in which the two light receiving elements are disposed spaced apart from each other.

(3)

The sensor unit according to (1) or (2), in which the two light receiving elements are each formed in a quadrangular shape in a top view, and are disposed such that one side of each light receiving element is parallel to a tangent to the circle concentric with the light emitting element.

(4)

The sensor unit according to any one of (1) to (3), in which
the light emitting element and the two light receiving elements are disposed on the same plane, and
the transmissive window is provided to be slanted with respect to a straight line connecting the light emitting element and one of the two light receiving elements and another straight line connecting the light emitting element and the other of the two light receiving elements.
   (5)

The sensor unit according to any one of (1) to (4), in which each of the two light receiving elements is disposed such that a center portion of each light receiving element is positioned on the circle concentric with the light emitting element.

(6)

The sensor unit according to (4), in which each of the two light receiving elements is disposed at the same distance from the light emitting element.

(7)

The sensor unit according to (4) or (6), in which the transmissive window is provided such that a window surface of the transmissive window is parallel to a straight line connecting the two light receiving elements.

(8)

The sensor unit according to any one of (4) to (7), in which the transmissive window is slanted such that reflected light emitted from the light emitting element and reflected from the transmissive window is incident on a region other than a region where the two light receiving elements are disposed.

(9)

A toilet seat device including:
a sensor unit; and
a toilet seat provided with the sensor unit, in which
the sensor unit includes:
a light emitting element that emits light toward an object,
two light receiving elements that receive light from the object, and
a transmissive window that is disposed between the light emitting element and the object and transmits light emitted from the light emitting element, and
the two light receiving elements are disposed so as to be positioned on a circle concentric with the light emitting element and to be positioned in a range of less than 180 degrees around the light emitting element.
   (10)

The toilet seat device according to (9), in which
the light emitting element and the two light receiving elements are disposed on the same plane, and
the transmissive window is provided to be slanted with respect to a straight line connecting the light emitting element and one of the two light receiving elements and another straight line connecting the light emitting element and the other of the two light receiving elements.

Further effects and modifications can be easily derived by a person having skill in the art. Thus, broader aspects of the present invention are not limited to the specific details and representative embodiments illustrated and described above. Accordingly, various modifications may be made without departing from the spirit or scope of the general inventive concept as defined by the appended claims and their equivalents.

What is claim is:

1.-8. (canceled)

9. A toilet seat device comprising:
a sensor unit; and
a toilet seat provided with the sensor unit, wherein
the sensor unit includes:
a light emitting element that emits light toward an object,
two light receiving elements that receive light from the object, and
a transmissive window that is disposed between the light emitting element and the object and transmits light emitted from the light emitting element, and
the two light receiving elements are disposed so as to be positioned on a circle concentric with the light emitting element and to be positioned in a range of less than 180 degrees around the light emitting element.

10. The toilet seat device according to claim 9, wherein
the light emitting element and the two light receiving elements are disposed on the same plane, and
the transmissive window is provided to be slanted with respect to a straight line connecting the light emitting element and one of the two light receiving elements and another straight line connecting the light emitting element and the other of the two light receiving elements.

11. The toilet seat device according to claim 9, wherein the two light receiving elements are disposed spaced apart from each other.

12. The toilet seat device according to claim 9, wherein the two light receiving elements are each formed in a quadrangular shape in a top view, and are disposed such that one side of each light receiving element is parallel to a tangent to the circle concentric with the light emitting element.

13. The toilet seat device according to claim 9, wherein each of the two light receiving elements is disposed such that a center portion of each light receiving element is positioned on the circle concentric with the light emitting element.

14. The toilet seat device according to claim 10, wherein each of the two light receiving elements is disposed at the same distance from the light emitting element.

15. The toilet seat device according to claim 10, wherein the transmissive window is provided such that a window surface of the transmissive window is parallel to a straight line connecting the two light receiving elements.

16. The toilet seat device according to claim 10, wherein
the transmissive window is slanted such that reflected light emitted from the light emitting element and reflected from the transmissive window is incident on a region other than a region where the two light receiving elements are disposed.
